(19) United States
(12) Patent Application Publication
(10) Pub. No.: US 2006/0172923 A1
(43) Pub. Date: Aug. 3, 2006

(54) Method of activating insulin receptor substrate-2 to stimulate insulin production

(75) Inventors: Gerhard Seipke, Hofheim (DE); Irini Rakatzi, Dusseldorf (DE); Olaf Dransfeld, Dusseldorf (DE); Juergen Eckel, Erkrath (DE)

Correspondence Address: ROSS J. OEHLER;SANOFI-AVENTSI U.S. LLC, 1041 ROUTE 202-206, MAIL CODE: D303A, BRIDGEWATER, NJ, 08807, US

(73) Assignee: Sanofi-Aventis Deutschland GmbH, Frankfurt (DE)

(21) Appl. No.: 11/398,869

(22) Filed: Apr. 6, 2006

Related U.S. Application Data
(63) Continuation of application No. 11/179,972, filed on Jul. 11, 2005, now abandoned.
Application No. 11/179,972 is a continuation of application No. 10/298,496, filed on Nov. 18, 2002, now abandoned.
(60) Provisional application No. 60/331,510, filed on Nov. 19, 2001.

(51) Int. Cl. A61K 38/28

(52) U.S. Cl. 514/3

(57) ABSTRACT

The invention relates to new methods and compositions for treating diabetics, pre-diabetics, and patients at risk of becoming diabetic or with impaired glucose tolerance. The invention, in one embodiment, involves activating insulin receptor substrate-2 to protect against loss of beta cell mass, protect against loss of beta cell function, rejuvenate beta cells mass, rejuvenate beta cell function or any combination thereof, thereby stimulate insulin production using an effective amount of LyS^{B3},Glu^{B29} insulin to patients in need of this treatment.

The invention relates to new methods and compositions for treating diabetics, pre-diabetics, and patients at risk of becoming diabetic or with impaired glucose tolerance. The invention, in one embodiment, involves activating insulin receptor substrate-2 to protect against loss of beta cell mass, protect against loss of beta cell function, rejuvenate beta cells mass, rejuvenate beta cell function or any combination thereof, thereby stimulate insulin production using an effective amount of LyS^{B3},Glu^{B29} insulin to patients in need of this treatment.

### BACKGROUND OF THE INVENTION

Insulin therapy of diabetic patients aims to achieve tight blood glucose control in order to reduce the progression of long-term complications (1). However, the pharmacokinetic characteristics of currently available insulin preparations are unable to mimick the pattern of endogenous insulin secretion and make it impossible to achieve sustained normoglycemia (2). Great efforts have been made to develop novel insulin molecules with altered pharmacodynamic characteristics that might lead to an improved glycemic control using recombinant DNA technology (for review, see 3-5). One limiting, factor is the slow absorption of conventional unmodified insulin from subcutaneous tissues due to the slow dissociation rate of hexameric insulin complexes into monomers at the injection site (6,7). Modification of the B26-B30 region of the insulin molecule, particularly substitution of amino acids with charged residues at the association sites, allows the production of a range of insulin analogs with reduced self association exhibiting no profound perturbations of insulin receptor recognition (4,8). This has been demonstrated for insulin analogs such as Lispro (Lys^{B28},Pro^{B29}) insulin and insulin aspart (Asp^{B28} insulin), two rapid acting insulins that are in clinical use and were both found to improve postprandial glycemic control (3,5).

A major concern related to the long-term use of insulin analogs stems from the observation that modifications of the insulin molecule in the B10 and B26-B30 region alter the affinity for the IGF-I receptor more than for the insulin receptor, and may lead to an enhanced mitogenic activity of these analogs (9). This potential safety risk was first recognized for the analog Asp^{B10} insulin, that was found to exhibit a tumor-promoting activity in Sprague-Dawley rats (10) and turned out to induce a profound mitogenic effect in many cell systems (11-13). The enhanced mitogenic signaling profile of an insulin analog may result from i) an increased affinity towards the IGF-I receptor resulting in an attenuated IGF-I receptor signaling (9), ii) the so called timing-dependent specificity that describes a distinct correlation between the mitogenic potential and the occupancy time at the insulin receptor for a given insulin analog (14), and iii) a combination of both IGF-I and insulin receptor mediated processes. Most recent data suggest that the mitogenic properties correlate better with IGF-I receptor affinities than with insulin receptor off-rates (12). Consistently, the increased mitogenic potency and the potential carcinogenic effect of prolonged exposure to high doses of Asp^{B10} insulin was shown to result from the stimulation of the IGF-I receptor (15).

In the present investigation, the signaling properties of two novel rapid acting insulin analogs, Lys^{B3},Glu^{B29} insulin (HMR 1964) and Lys^{B3},Ile^{B28} insulin (HMR 1153) have been analyzed in comparison to native human insulin and the analog Asp^{B10} insulin using rat and human myoblasts and differentiated muscle cells. Methods of making these analogs and other analogs are described in U.S. Pat. No. 6,221,633, which is hereby incorporated by reference. Attempts have been made to correlate the mitogenic potential of the analogs to i) the initial receptor binding and processing, ii) the activation of the Shc/MAP-kinase pathway, and iii) the induction of the tyrosine phosphorylation of IRS-½. The data clearly show that HMR 1964 and 1153 activate highly divergent signaling patterns in a fashion independent of their binding affinities for the IGF-I receptor. In contrast to 1153, HMR 1964 is able to exclusively activate the IRS-2 pathway, both in myoblasts and differentiated muscle cells. In human skeletal muscle cells, 1964 activated IRS 2 to a greater extent than did regular human insulin. Thus, in one embodiment, HMR 1964 activates IRS 2 in vivio to a greater extent than human insulin. Thus, the receptor phosphorylation and/or processing is an additional determinant of signaling specificity of the insulin molecule.

In one embodiment, IRS 2 activation may be a factor in maintaining viability of the beta cell in the pancreas (herein refered to as beta cells) and thus maintaining insulin secretion in states in which beta cell health is jeopardized such as type 1 diabetes, insulin resistant non diabetic states (obesity, IGT) and finally in type 2 diabetes. For example, the phenotype of the transgenic IRS 2 KO mouse demonstrates that the lack of IRS 2 leads to beta cell loss and the development of diabetes. Thus, for example, an IRS 2 activator may have potential therapeutic value in certain disease states as described

### SUMMARY OF THE INVENTION

The present invention involves, in one embodiment, a method of activating insulin receptor substrate-2 (IRS-2) to stimulate insulin production, which comprises administering to a patient in need thereof an effective amount of LyS^{B3},Glu^{B29} insulin. In one embodiment, IRS-2 activation to stimulate insulin production is a long term effect. More specificly, IRS-2 activation may be a way of protecting against loss of beta cell mass, protecting against loss of beta cell function, rejuvinating beta cells mass or rejuvinating beta cell function or any combination thereof, which in returns leads to insulin production. In one embodiment, this includes substantially recovering full functionality of beta cells. As used herein protect means to maintain, increase or maintain and increase beta cell function.

In one embodiment, beta cell function as defined herein is measured by the production of insulin. In another embodiment, a test model may be used to determine if beta cell depletion is inhibited. For example, Zucker Diabetic Fatty (ZDF) rats are a model of the human phenotype of type 2 diabetes. These animals evolve through a prediabetic stage with obesity and insulin resistance followed by the development of type 2 diabetes. During the insulin resistant, obese non diabetic phase animals develop hyperplasia of beta cells with a concomittant increase in insulin secretion with maintance of normoglycemia. As the animals progress in their disease process, decreases in beta cell mass are associated with a reduction in insulin secretion and the development of overt hyperglycemia and diabetes. In the prediabetic, obese, insulin resistant animals, as well as the overtly diabetic animals, an increase in beta cell death by an apoptotic mechanism occurs (Shimabukuro M et al. PNAS 95:2498-2502; Pick A, et al. Diabetes 47: 358-364; Finegood D et al. Diabetes 50:1021-1029, 2001). Thus, the ZDF rat may be used as a preclinical model in which the anti-apoptopic, cytoprotective effect of agents acting directly on beta cells to prevent apoptosis can be assessed. For example, animals administered a test substance exerting an anti-apoptotic cytoprotective effect on beta cells may delay and/or prevent the increase in beta cell apoptosis as assessed by DNA fragmentation and the attendant reduction in beta cell mass as assessed by histomorphometric analysis. The cytoprotective beta cell effects of a test agent systemically administered to ZDF animals in the prediabetic phase of their disease may also be manifested by a delay in the onset of loss of beta cell function as assessed by reductions in insulin secretion in reponse to hyperglycemia or to a delay in the onset of overt diabetes manifested by fasting hyperglycemia.

Patients receiving this treatment can include those with insulin resistance indicated by elevated plasma insulin levels in the absence of any impairment in glucose metabolism, impaired glucose tolerance and/or has impaired fasting glucose (IFG). Also included within the scope of the treatment are patients with subclinical beta cell autoimmune disease, type I or type II diabetics or patients having at least a reduced ability to produce insulin because their beta cells are impaired. In another embodiment, patients with autoimmune problems or those suffering from obesity, insulin resistance, and/or hyperinsulinemic are susceptible to treatment according to the present invention.

In addition to Lys^{B3},Glu^{B29} insulin (HMR 1964) for use in the compositions and methods of the invention, as described herein, homologs of this insulin analog which posess at least one of the following properties chosen from preferential activation of IRS 2, protecting against loss of beta cell mass, protecting against loss of beta cell function, rejuvinating beta cells mass or rejuvinating beta cell function or any combination thereof may also be useful in the practice of the invention. Preferential activation of IRS 2, as used herein, is the ability of the insulin analog to activate IRS 2 more effectively than IRS 1 and/or the ability of the insulin analog to activate IRS 2 to a greater extent that human insulin.

The similarity between HMR 1964 and different insulin analogs can be expressed by the degree of homology between the protein sequence. 50% homology means, for example, that 50 out of 100 amino acid positions in the sequences correspond to each other. The homology of proteins is determined by sequence analysis. Thus, the present invention also relates to insulin homologs which have a degree of homology to the amino acid sequence of HMR 1964 of at least about 50%, for example, at least about 60%, 70%, 75%, 80%, 85%, 90%, and 95%. Homology as used herein is defined as a sequence modified with substitutions, insertions, deletions, and the like.

One embodiment of the invention is a method of protecting beta cell mass of a patient, which comprises administering to the patient an effective amount of LyS^{B3},Glu^{B29} insulin. The patient may, for example be a Type II diabetic. A Type II diabetic may, for example, be impaired glucose tolerant and/or have impaired fasting glucose. In one embodiment, the ability of the beta cells of the patient to produce insulin may have been impaired. Also within the practice of the invention is when the patient has autoimmune deficiencies and/or is obese, insulin resistant, and/or hyperinsulinemic.

The invention also includes: a method of protecting of beta cell function of a patient, which comprises administering to the patient an effective amount of LyS^{B3},Glu^{B29} insulin; a method of rejuvinating beta cells mass of a patient, which comprises administering to the patient an effective amount of LyS^{B3},Glu^{B29} insulin; a method of rejuvinating beta cell function of a patient, which comprises administering to the patient an effective amount of Lys^{B3},Glu^{B29} insulin; and a method of activating insulin receptor substrate-2 to protect at least one property chosen from beta cell mass and beta cell function, which comprises administering to a patient an effective amount of Lys^{B3} Glu^{B29} insulin.

Another embodiment of the invention is a pharmaceutical composition comprising comprising Lys^{B3},Glu^{B29} insulin in an amount effective to protect against loss of beta cell mass, protect against loss of beta cell function, rejuvenate beta cells mass or rejuvenate beta cell function or any combination thereof without corresponding signficant reduction in blood glucose levels.

Further embodiments of the invention include: a pharmaceutical composition consisting essentially of Lys^{B3},Glu^{B29} insulin, wherein said HMR 1964 is present in an amount ranging from about 0.01 IU/kg to about 0.1 IU/kg; and a pharmaceutical composition comprising LyS^{B3},Glu^{B29} insulin with the provisio that said pharmaceutical composition does not contain human insulin, wherein said HMR 1964 is present in an amount ranging from about 0.01 IU/kg to about 0.1 IU/kg.

The compositions and methods of the invention may also be used as part of a combination therapy. For example the compositions of the invention may be administered with with human insulin, insulin secretagogues, and/or other additives know in the art, such as, for example, thiazolidinediones, metformin, acarbose, sulfonylureas, and glitazones.

### BRIEF DESCRIPTION OF THE DRAWINGS

FIG. 1. Autophosphorylation of the IGF-I receptor in K6 myoblasts in response to insulin and insulin analogs. Myoblasts were stimulated for 10 min with human insulin (HI) or the indicated analogs at a concentration of 500 nmol/l, and the IGF-I receptor was immunoprecipitated (IP) as described. The immunopellet was analyzed by SDS-PAGE and immunoblotted (IB) with anti-phosphotyrosine (pY) antibodies using the ECL system. Quantification was performed on a Lumilmager work station. Results are expressed relative to the basal value and are mean values±SEM of three separate experiments. Significantly different from human insulin, *p<0.006; #p<0.001

FIG. 2. Coprecipitation of Shc proteins with the IGF-I receptor after stimulation of K6 myoblasts with insulin or insulin analogs. Myoblasts were stimulated with human insulin (HI) or analogs and the IGF-I receptor was immunoprecipitated, as described in FIG. 1. Immunopellets were processed and immunoblotted with anti-Shc antibodies. Quantification of the 66 kDa protein band was performed using the Lumilmager system and is presented in the lower panel. Data are mean values±SEM of three separate experiments.

FIG. 3. Tyrosine phosphorylation of Shc proteins in K6 myoblasts in response to insulin and insulin analogs. Cells were stimulated with the peptide hormones and the Shc proteins were immunoprecipitated as described. Immunopellets were processed and immunoblotted with anti-phosphotyrosine antibodies, as outlined in FIG. 1. Equal loading was ensured by reprobing the stripped filters with anti-Shc antibodies. The 52 and the 66 kDa Shc protein band was quantified using the Lumilmager system. Data represent mean values±SEM of five separate experiments.

FIG. 4. Activation of p42/44 MAP kinase by insulin and insulin analogs in K6 myoblasts. Cells were stimulated with the different insulins as described in FIG. 1 and lysed. Cellular proteins were separated by SDS-PAGE and immunoblotted with phospho-ERK½ antibodies, stripped and reprobed with ERK½ antibodies using ECL detection. Phospho-ERK½ signals were quantified using the Lumilmager software. Data are mean values±SEM obtained from four separate experiments. *Significantly different from basal and all other stimulated values with at least p<0.05

FIG. 5. Effects of insulin, insulin analogs and IGF-I on the incorporation of 5-bromo-2′-deoxyuridine (BrdU) into DNA in K6 myoblasts. Myoblasts were serum-starved for 30 h in DMEM and subsequently incubated with BrdU in the absence (basal) or presence of the indicated concentrations of peptide hormones or fetal calf serum (FCS) for 16 h. Cells were fixed, denatured and the incorporation of BrdU was determined using an anti-BrdU antiserum and ECL detection. Data are mean values±SEM of four separate experiments.

FIG. 6. Tyrosine phosphorylation of IRS proteins in K6 myoblasts in response to insulin and insulin analogs in K6 myoblasts. Cells were stimulated as outlined in FIG. 1 and both IRS-1 and IRS-2 were immunoprecipitated and processed for immunoblotting with anti-phosphotyrosine antibodies. Filters were stripped and reprobed with anti-IRS-1 or anti-IRS-2 antiserum, respectively, to ensure equal loading. Signals were quantified using Lumilmager software. The data shown are mean values±SEM of 3-4 separate experiments. *Significantly different from basal and all other stimulated values (p<0.05); #significantly different from HI and 1964 (p<0.05).

FIG. 7. Tyrosine phosphorylation of IRS proteins in proliferating human skeletal muscle cells in response to insulin and insulin analogs. Human myoblasts (10⁶ cells/dish) were cultured as described and subjected to serum starvation for 4 days. The cells were then stimulated with the different insulins as described in FIG. 1. IRS-1 and IRS-2 were immunoprecipitated and processed for immunoblotting with anti-phosphotyrosine antibodies. Stripping, reprobing with anti-IRS-1 and anti-IRS-2 antibodies, and quantification of the signals was performed as described in FIG. 6. Data are mean values±SEM of 4-5 separate eperiments. *Significantly different from basal and all other stimulated values (p<0.05); #significantly different from human insulin (p<0.05).

FIG. 8. Tyrosine phosphorylation of IRS proteins in adult rat cardiomyocytes in response to insulin and insulin analogs. Freshly isolated cardiomyocytes (4×10⁵ cells) were stimulated for 10 min with 500 nmol/l of insulin and insulin anlogues. Cells were then lysed with RIPA buffer and processed for immunoprecipitation and immunoblotting of IRS-½ as described in FIG. 6. Quantification was performed using Lumilmager software. Data are mean values±SEM of 4 separate experiments. *Significantly different from basal and all other stimulated values (p<0.05); #significantly different from HI and 1964 (p<0.05).

FIG. 9. Transport of 3-O-methylglucose in adult cardiomyocytes in response to insulin and HMR 1964. 4×10⁵ cells/ml were incubated for 10 min in the absence (basal) or presence of the indicated concentrations of insulin or HMR 1964. Initial rates of 3-O-methylglucose were then determined over a 10-s assay period as outlined in the Methods section. Data are mean values±SEM of 3-4 separate experiments.

### DETAILED DESCRIPTION OF THE INVENTION

The potentially enhanced mitogenic activity of insulin analogs may affect the safety profile of the human hormone and requires a detailed analysis of any new analog considered for therapeutic applications. The signaling properties and the mitogenic potency of two novel rapid acting insulin analogs, Lys^{B3},Glu^{B29} insulin (HMR 1964) and LyS^{B3},Ile^{B28} insulin (HMR 1153) have been assessed in comparison to native human insulin and the analog Asp^{B10} insulin using rat and human myoblasts and differentiated muscle cells. In K6 myoblasts expressing a high level of IGF-I receptors, both binding and internalization were 2-3fold higher for Asp^{B10} insulin and HMR 1153 when compared to HMR 1964 and regular insulin. This correlated with a prominent Shc/IGF-I receptor interaction, tyrosine phosphorylation of Shc, activation of ERK1 and ERK2, and stimulation of DNA synthesis by HMR 1153 and Asp^{B10} insulin.

In contrast, HMR 1964 produced a marginal activation of the Shc/MAP kinase cascade and was equipotent to insulin in stimulating DNA synthesis in K6 myoblasts. In these cells HMR 1964 produced a minor activation of IRS-1 tyrosine phosphorylation despite a significantly higher autophosphorylation of the IGF-I receptor when compared to insulin. However, this analog produces a prominent activation of IRS-2 with a significantly stronger effect than insulin in human myoblasts. Preferential activation of IRS-2 was also observed in differentiated cardiomyocytes where HMR 1964 increased 3-O-methylglucose transport to the same extent as human insulin. Thus i) the mitogenic properties of insulin analogs may result from a complex series of initial receptor interactions including internalization and phosphorylation, ii) the primary structure of the insulin molecule may be sufficient to control hormonal action at the downstream level, and iii) the mitogenic potential of HMR 1964 is identical to that of insulin and that selective activation of IRS-2 by this analog may open new avenues for optimized insulin therapy.

For example, IRS-2 activation by HMR 1964 may be a way of protecting against loss of beta cell mass, protecting against loss of beta cell function, rejuvinating beta cells mass or rejuvinating beta cell function or any combination thereof, which in returns leads to insulin production.

In one embodiment, a protective or rejuvinatory effect to beta cells will not likely be observed upon a first administration of HMR 1964. One of skill in the art will recognize that the amount of time necessary will depend on the extent of beta cell damage present and the amount of HMR 1964 administed. For example, regular administration for one week or more, such as two weeks or three weeks may be necessary, as may administration for one month or greater, such as, for example, two months, three months or greater.

The amount of HMR 1964 administed will of course depend on the disease state being treated and/or the amount of damage to beta cell function and mass. For example, in one embodiment, HMR 1964 is administered in an amount effective to protect against loss of beta cell mass, protect against loss of beta cell function, rejuvenate beta cells mass or rejuvenate beta cell function or any combination thereof without corresponding signficant reduction in blood glucose levels. Corresponding, as used in this context, refers to a signficant reduction in blood glucose levels that occurs after administration of HMR 1964, for example, immediately after, one hour, two hours, three hours, 6 hours, or 12 hours following administration.

In one embodiment, HMR 1964 is administered in an amount effective to deliver HMR 1964 to the pancreas but not substantially lower blood glucose levels. As used herein, substantially lowering blood glucose levels refers to a therapeutically effective lowering of blood glucose levels. In another embodiment, HMR 1964 may be administered in an amount effective to deliver HMR 1964 to the pancreas for a protective or rejuvenatory effect yet still substantially lower blood glucose levels.

In one embodiment, the amount administered is at the upper limit below the amount of insulin normally administered to a diabetic. For example, per day, less than about 2 IU/kg, such as, for example, about 1.5 IU/kg, about 1.0 IU/kg, about 0.5 IU/kg, about 0.25 IU/kg, and about 0.1 IU/kg or less. The lower limit may, for example, in one embodiment, be about 0.01 IU/kg or greater, such as, for example, about 0.025 IU/kg, about 0.05 IU/kg, about 0.07 IU/kg, about 0.08 IU/kg, and about 0.09 IU/kg or greater. Low amounts of HMR 1964 may be administed in a combination therapy with other insulins, such as human insulin. Thus, in one embodiment, a pharmaceutical composition of the invention is a pharmaceutical composition consisting essentially of HMR 1964, wherein said HMR 1964 is present in an amount ranging from about 0.01 IU/kg to about 0.1 IU/kg or a pharmaceutical composition comprising HMR 1964 with the provisio that said pharmaceutical composition does not contain human insulin, wherein said HMR 1964 is present in an amount ranging from about 0.01 IU/kg to about 0.1 IU/kg.

### Research Design and Methods

### Materials

Native human insulin and the insulin analogs Lys^{B3},Glu^{B29} insulin (HMR 1964), Lys^{B3}, Ile^{B28}(HMR 1153) and Asp^{B10} insulin as well as the ¹²⁵I-labeled insulin preparations (specific activity 260 mCi/mg) were provided by Aventis Pharma GmbH (Frankfurt, Germany). 3-O-[¹⁴C]Methyl-D-glucose and L-[1-¹⁴C]glucose were purchased from Amersham Pharmacia Biotech (Freiburg, Germany). Reagents for SDS-PAGE were supplied by Amersham Pharmacia Biotech and Sigma (Deisenhofen, Germany). Collagenase was from Serva (Heidelberg, Germany) and bovine serum albumin (BSA, Fraction V, fatty acid free) was obtained from Boehringer Mannheim (Germany). Protein A-trisacryl (GF-2000) and protein G-agarose were products from Pierce (Oud Beijerland, The Netherlands). The monoclonal IGF-I receptor antibody was purchased from Oncogene research products (Cambridge, Mass., USA). The polyclonal anti-SHC, anti-IRS 1 and anti-IRS 2 antibodies were obtained from Biomol (Hamburg, Germany). IRS1 and IRS 2 antisera used for immunoprecipitation were kindly provided by Dr. J. A. Maassen (Leiden, The Netherlands). The phosphospecific p42/44 MAP-kinase antisera (Thr202/Tyr204) and the p44/42 MAP-kinase antibodies were products of New England Biolabs (Schwalbach/Taunus, Germany). The anti-phosphotyrosine antiserum RC20 was produced by Becton Dickinson (Heidelberg, Germany). Horseradish peroxidase conjugate (anti-rabbit IgG) as the secondary antibody for ECL was purchased from Promega (Mannheim, Germany). Stripping solution was a product of Alpha Diagnostics (San Antonio, Tex., USA). The cell proliferation ELISA chemiluminescence kit was purchased from Boehringer (Mannheim, Germany). Fetal calf serum, Dulbecco's modified Eeagle medium (DMEM), non-essential amino acids and penicillin/streptomycin were provided from Gibco (Eggenstein, Germany). Primary human skeletal muscle cells, basal medium and supplement pack for growth medium were obtained from PromoCell (Heidelberg, Germany). All other chemicals were of the highest grade commercially available.

### Cell Culture and Isolation of Cardiomyocytes

K6 myoblasts represent a rat heart muscle cell line that was established and characterized (16). These cells are insulin-sensitive and express the glucose transporter GLUT4 (16). Cells were kept in monolayer culture in DMEM supplemented with 10% fetal calf serum, non essential amino acids (1%), streptomycin (100 μg/ml) and penicillin (100 U/ml) in 175 cm² flasks in an atmosphere of 5% CO₂ at 37° C. Myoblasts were maintained in continuous passages by trypsinization of subconfluent cultures 7 days after plating. The medium was changed every 72 h. Cell number was determined after cell dissociation with trypsin/EDTA at 37° C.

Primary human skeletal muscle cells obtained from satellite cells isolated from M. rectus abdominis of a 28 year old male Caucasian donor were supplied as proliferating myoblasts. These cells were kept in skeletal muscle cell growth medium (basal medium containing: fetal calf serum, 5%; epidermal growth factor, 10 ng/ml; basic fibroblast growth factor, 1 ng/ml; fetuin, 0.5 mg/ml; insulin, 0.1 mg/ml; dexamethasone, 0.4 μg/ml; gentamicin, 50 μg/ml; and amphotericin B, 50 ng/ml) for two population doublings. Cells were then frozen and stored in liquid nitrogen until further use. For stimulation experiments, 10⁶ cells/dish were seeded in growth medium and cultured for 2 days. Cells were then washed with PBS and cultured for 4 days in the absence of serum and insulin. The cells were then cultured for 1 h with fresh medium containing 0.5% BSA, and subsequently stimulated with the hormones.

Adult rat cardiomyocytes were isolated by perfusion of the heart with collagenase, as previously described by us (17). Male Wistar rats (280-340 g) were used in all experiments. The final cell suspension was incubated for 60 min until further use in HEPES buffer (130 mM NaCl, 4.7 mM KCl, 1.2 mM KH₂PO₄, 25 mM HEPES, 5 mM glucose, 2% (w/v) bovine serum albumin, pH 7.4, equilibrated with oxygen) containing MgSO₄ and CaCl₂ (final concentrations: 1 mM) at 37° C. in a rotating waterbath shaker. The cell viability was judged by determination of the percentage of rod-shaped cells and averaged 90-97% under all incubation conditions.

### Binding, Internalization and Degradation

For binding studies myoblasts were suspended in DMEM containing 10% fetal calf serum (FCS) and seeded in 6 well culture dishes at a density of 2×10⁵ cells/well. After 24 h in culture the cells were washed 2 times with phosphate-buffered saline (PBS) and incubated for 60 min at 37° C. in DMEM without FCS containing 2% bovine serum albumin (BSA). ¹²⁵I-labeled human insulin or one of the ¹²⁵I-labeled insulin analogs (0.1 μCi, 5×10⁻¹¹ M) was then added along with the corresponding unlabeled peptide hormone (10⁻⁸ M), and incubation was continued for 10 min at 37° C. The medium was then removed, the cells were washed twice and lysed with 0.1% sodium dodecylsulfate (SDS) and the radioactivity was determined in a gamma counter. Non specific binding was measured in parallel incubations performed in the presence of an excess of the corresponding unlabeled hormone (10⁻⁵ M), respectively. All assays were performed in triplicate. Internalization was determined after incubation of myoblasts for 60 min at 37° C. using a concentration of 5×10⁻¹¹ M (0.1 μCi) of the different insulin molecules. Unbound insulin was first removed by washing the cells with cold PBS, followed by washing the cells three times with cold PBS at acid pH (pH 2.75, 0.1% BSA). Cells were lyzed in 1% SDS/0.1 N NaOH, and the remaining radioactivity was determined in a gamma counter. The same incubation conditions were also used for determination of the degradation of the insulin molecules. Briefly, after 60 min aliquots of the supernatant were subjected to trichloroacetic acid (TCA) precipitation and the degradation was calculated from the increase in TCA solubility of the tracer, as outlined earlier (18).

### Immunoprecipitation

K6 myoblasts (3×10⁶) were plated in their regular growth medium. After a 24 h culture period the medium was removed and replaced with fresh medium without FCS containing 0.5% BSA. Following a 2 h incubation at 37° C. cells were stimulated with human insulin or one of the insulin analogs (final concentration 5×10⁻⁷ M) for 10 min. After washing twice with ice-cold PBS, cells were incubated in RIPA lysis buffer (50 mM Tris-HCL (pH 7.4), 1% NP-40, 0.25% Na-deoxycholate, 150 mM NaCl, 1 mM EDTA, 1 mM PMSF, 1 μg/ml each aprotinin, leupeptin, pepstatin, 1 mM Na₃VO₄ and 1 mM NaF) for 2 h at 4° C. with gentle agitation. Human myoblasts were cultured as outlined above and stimulated with the insulin molecules for 10 min followed by lysis with RIPA buffer. Freshly isolated cardiomyocytes were preincubated in a rotating water-bath shaker according to our protocols (19), and after a 10 min stimulation with the different insulin analogs the lysis was performed using RIPA buffer. For immunoprecipitation cell lysate samples were then incubated with antibodies against the IGF-I receptor, IRS-1, IRS-2 or Shc at 4° C. and gently rocked overnight. The immunocomplexes were adsorbed on to Protein G-Sepharose or Protein A-Sepharose beads for 2 h at 4° C. during gentle agitation before being collected by centrifugation at 14,000 rpm for 30 s. Beads were washed 3 times with ice-cold PBS and used for Western blot analysis.

### Immunoblotting

The immunoadsorbed proteins were solubilized in Laemmli sample buffer and were resolved by SDS/PAGE on 8-18% (w/v) horizontal gradient gels, followed by transfer to polyvinylidene difluoride (PVDF) membranes. These were then blocked in TBS/Tween 0.05% plus 1% BSA for 1 h at room temperature and incubated with the appropriate primary antibody at 4° C. overnight. After extensive washing, membranes were incubated with horseradish peroxidase-conjugated secondary antibodies. Protein bands were then visualized by the enhanced chemiluminescence (ECL) method on a Lumilmager work station. MAP-kinase activation was assessed by immunobloting K6 cell lysates with phosphospecific ERK½ antibodies and the phosphorylated proteins were detected by the ECL method. The blots were stripped and reprobed with the polyclonal ERK½ antibody as described by the manufacturer. All blots were quantified using the Lumilmager software.

### DNA Synthesis and Determination of 3-O-Methylglucose Transport

For monitoring DNA synthesis, K6 myoblasts (1×10⁴ cells/well) were seeded in 24-well microtiter tissue culture plates and were cultured for 24 h in DMEM containing 10% FCS, followed by a 30 h culture period under serum-free conditions. Cells were then stimulated with the different peptide hormones or 10% FCS for 16 h with the simultaneous addition of 5-bromo-2′-deoxyuridine (BrdU). After removing the labeling medium, the cells were fixed and the DNA was denatured by addition of FixDenat (Boehringer Mannheim, Germany). Cells were then incubated with a peroxidase-conjugated anti-BrdU antiserum, and after addition of substrate the light emission was quantified on a Lumilmager work station.

The determination of 3-O-methylglucose transport using freshly isolated adult rat cardiomyocytes was performed at 37° C. in HEPES buffer (composition: 130 mM NaCl, 4.8 mM KCl, 1.2 mM KH₂PO₄, 1 mM MgCl₂, 1 mM CaCl₂, 25 mM HEPES, 5 mM glucose, 20 g/l BSA, pH 7.4). 4×10⁵ cells/ml were stimulated with insulin or insulin analogs for 10 min. The transport reaction was started by pipetting a 50 μl aliquot of the cell suspension to 50 μl of HEPES buffer containing 3-O-[¹⁴C]methyl-D-glucose (final concentration 100 μM). Carrier-mediated glucose transport was then determined using a 10-s assay period and L-[¹⁴C]glucose to correct for simple diffusion, as described in earlier reports from this laboratory (19,20).

### Statistical Analysis

All results are expressed as means±SEM. The significance of reported differences was evaluated by using the null hypothesis and t statistics for paired data. Corresponding sigificance levels are indicated in the figures.

### RESULTS

### Binding and Processing of Insulin and Insulin Analogs by K6 Myoblasts

It has been reported that modifications in the C-terminal region of the B-chain of insulin alter the affinity for the IGF-I receptor more than for the insulin receptor (21). As shown previously (13), cardiac myoblasts express a high level of IGF-I receptors with a marginal abundance of insulin receptors, thus representing a suitable tool to assess IGF-I receptor signaling by insulin analogs. As shown in Table 2, the analog HMR 1153 exhibited the highest binding to K6 myoblasts being comparable to the analog Asp^{B10} insulin, which has a reported higher affinity for the IGF-I receptor than regular insulin (22). In contrast the analog HMR 1964 showed a significantly lower binding potency that was similar to human insulin. It should be noted that these binding studies were performed at a high concentration of the peptide hormones (10⁻⁸ mol/l) in order to allow comparison to the studies on signal transduction and DNA synthesis described below. Interestingly, HMR 1964 also exhibited the lowest rate of internalization and low degradation by the myoblasts (Table 2). The data in Table 2 also show that internalization and degradation of an insulin molecule are not always directly correlated. Thus, human insulin showed a significantly lower rate of internalization compared to HMR 1153, but showed the highest degradation by K6 cells. This fits to the notion that insulin processing reflects a complex process involving internalization and degradation at different intracellular sites (23).

### Effect of Insulin and Insulin Analogs on the Shc/Map-Kinase Pathway

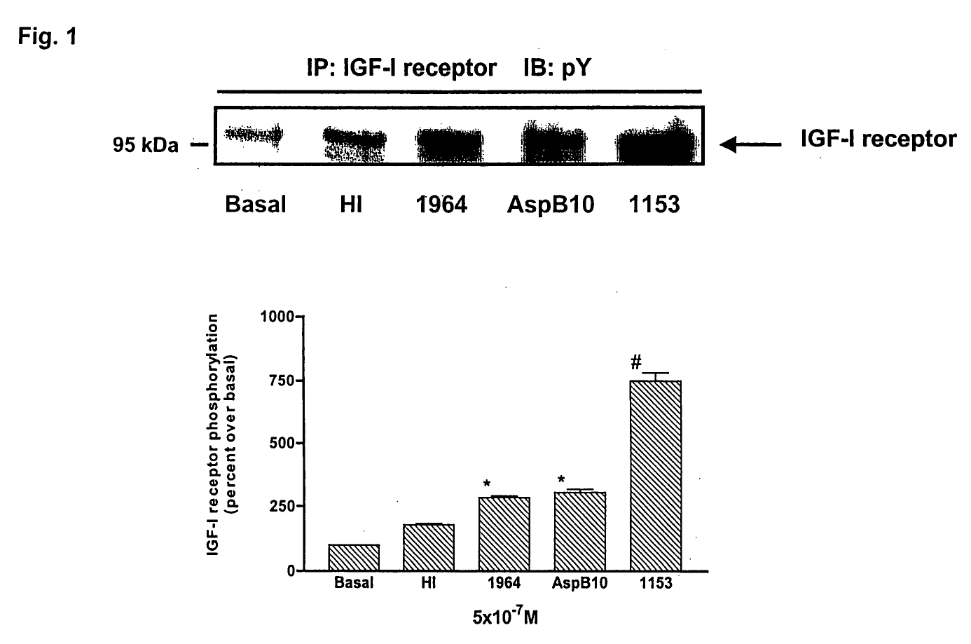

In order to assess the signaling potency of the different insulin analogs to the MAP-kinase pathway, the autophosphorylation of the IGF-I receptor, the phosphorylation of Shc proteins and the activation of p44/42 (ERK½) MAP-kinase was determined. As presented in FIG. 1, the analog HMR 1153 induced a very prominent autophosphorylation of the IGF-I receptor in the K6 myoblasts. This effect was about 2.5 fold higher than the autophosphorylation induced by Asp^{B10} insulin despite a comparable binding of these analogs (see Table 2). On the other hand, the analog HMR 1964 produced the same effect as Asp^{B10} insulin despite the lowest binding affinity to the K6 myoblasts. Thus, the level of receptor occupancy may not be sufficient to determine the signaling potency of an insulin molecule.

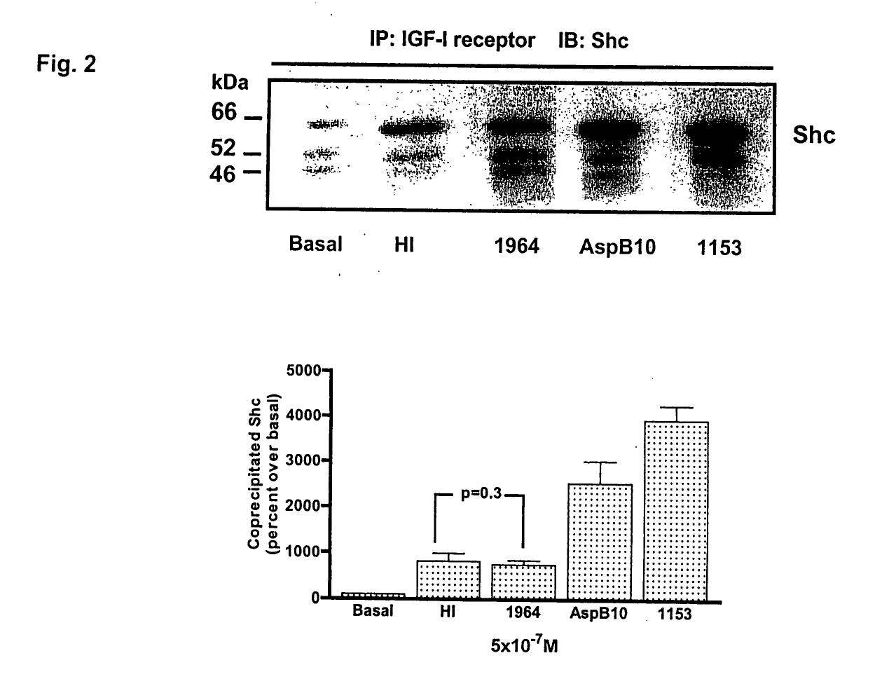

This is also shown in FIG. 2. Immunoprecipitates of the IGF-I receptor were immunoblotted with an anti-Shc-antibody that recognizes all three Shc-isoforms. These adaptor proteins play a central role in the activation of the MAP-kinase cascade (24). As can be seen from the data (FIG. 2), the 66 kDa Shc exhibited the most prominent association to the autophosphorylated IGF-I receptor in response to the insulin molecules. Asp^{B10} insulin and HMR 1153 induced a comparable association of the 66 kDa Shc to the IGF-I receptor that was about 3-4 fold higher compared to that induced by insulin and HMR 1964. Most importantly, no significant difference was observed between human insulin and HMR 1964 at this level of the signaling cascade. It should be noted that the much higher autophosphorylation of the IGF-I receptor by HMR 1153 does not lead to an appropriately strong interaction with Shc (FIG. 2). For all experiments equal loading was ensured by re-probing the blots with an anti-IGF-I-receptor-antibody (not shown in the Fig.).

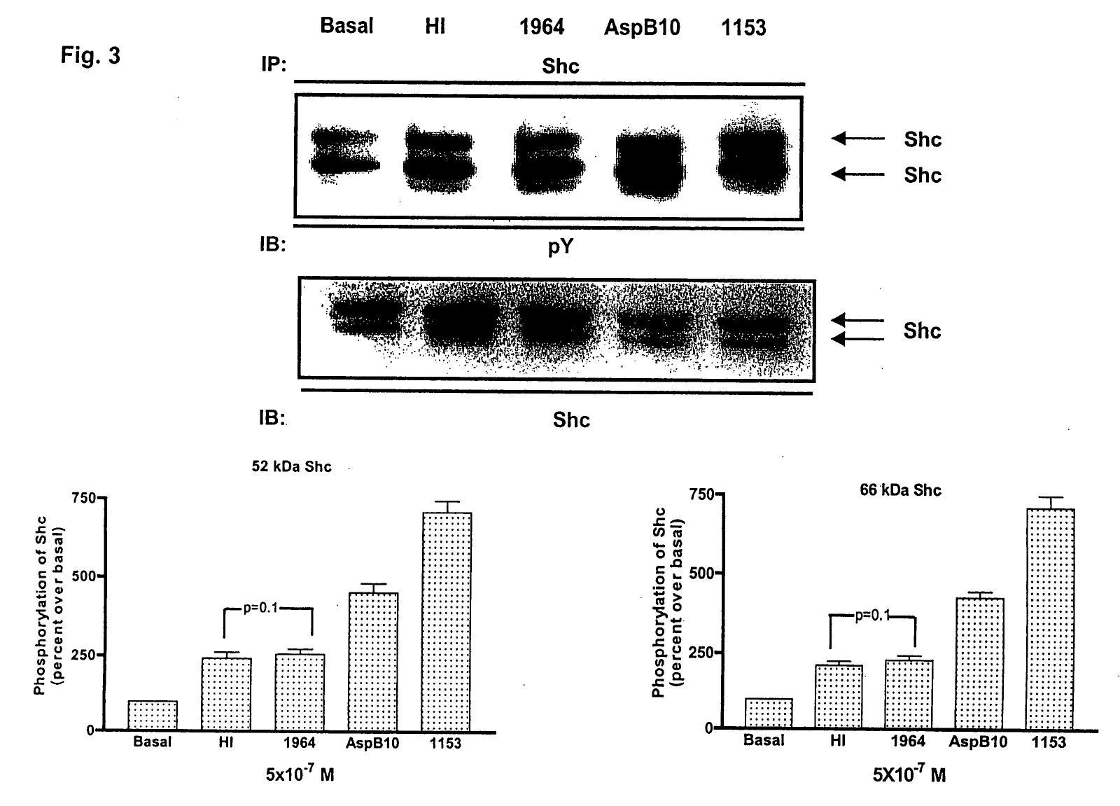

To further investigate the interaction between the IGF-I receptor and the Shc proteins, tyrosine phosphorylation of these intracellular substrates was determined after stimulation of K6 myoblasts with human insulin or the insulin analogs. For this assay the cell extracts were subjected to immunoprecipitation with an anti-Shc-antibody and the resulting precipitates were analyzed by immunoblotting with an anti-phosphotyrosine antiserum. As shown in FIG. 3, the K6 myoblasts express only two Shc proteins, the 66 kDa and the 52 kDa isoform. The two insulin analogs HMR 1153 and Asp^{B10} insulin induced the strongest tyrosine phosphorylation of the two Shc isoforms. Again, the analog HMR 1964 was comparable to human insulin inducing a much lower Shc phosphorylation (FIG. 3). Quantification of tyrosine phosphorylation of the most prominent 52 kDa Shc isoform from multiple experiments demonstrated a 7 fold increase in the level of Shc phosporylation after stimulation with HMR 1153 and 5 fold response after treatment with Asp^{B10} insulin. After stimulation with either HMR 1964 or human insulin an approximately 2 fold increase in Shc phosphorylation was observed (FIG. 3). The same results were obtained regarding the phosphorylation level of the 66 kDa Shc isoform (FIG. 3).

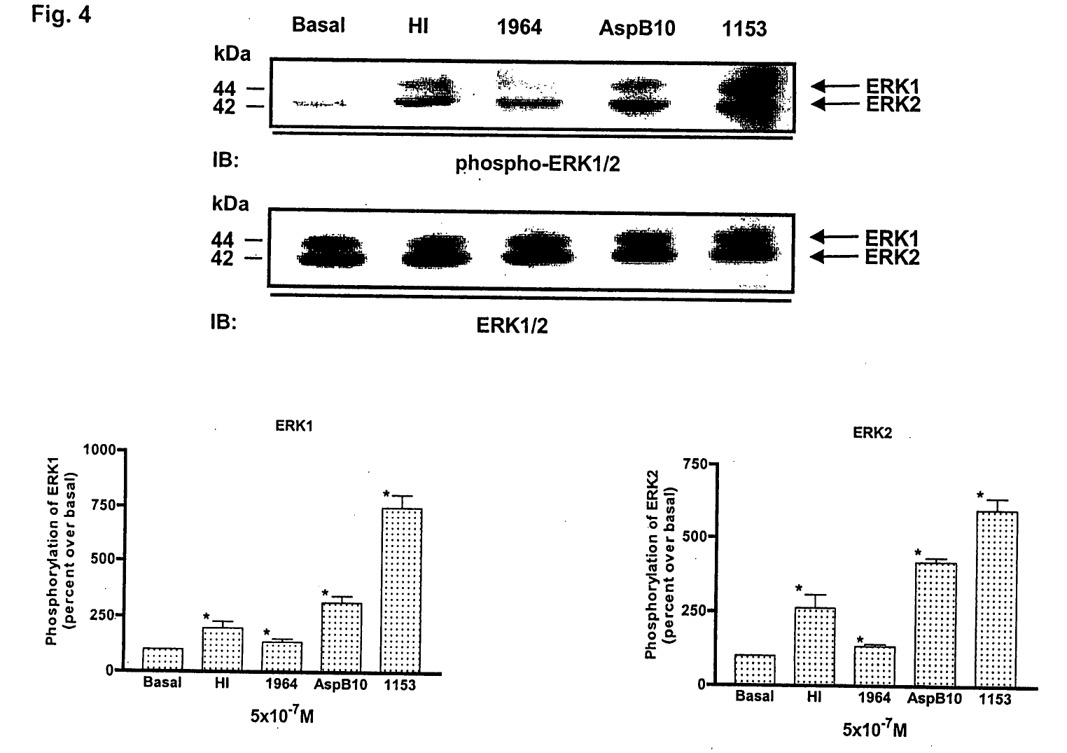

It has been reported that IGF-I receptor internalization regulates signaling via the MAP-kinase pathway but not the insulin receptor substrate-1 pathway (24). Taking into account a high rate of internalization of the analogs Asp^{B10} insulin and HMR 1153 and the stronger interaction with the Shc proteins, it was anticipated that these two analogs may induce a strong activation of the p42/44 MAP-kinase in the K6 myoblasts. Activation of the MAP-kinases was assessed by monitoring the phosphorylation state of these proteins using phospho specific MAP-kinase antiserum that detects tyrosine phosphorylated ERK1 and ERK2. The data shown in FIG. 4 indicate that K6 cells express both MAP-kinases, the 44 kDa and the 42 kDa isoform. The phosphorylation of both isoforms was strongly activated after stimulation of cells with either HMR 1153 or Asp^{B10} insulin. Quantification of the data showed a 7 and 5 fold activation for ERK1 and ERK2, respectively, after treatment with HMR 1153. Asp^{B10} insulin was less potent than HMR 1153 but still produced a significantly higher response compared to human insulin and HMR 1964. The latter analog also produced the lowest response of MAP-kinase activation that was significantly different from human insulin.

### DNA Synthesis in K6 Myoblasts

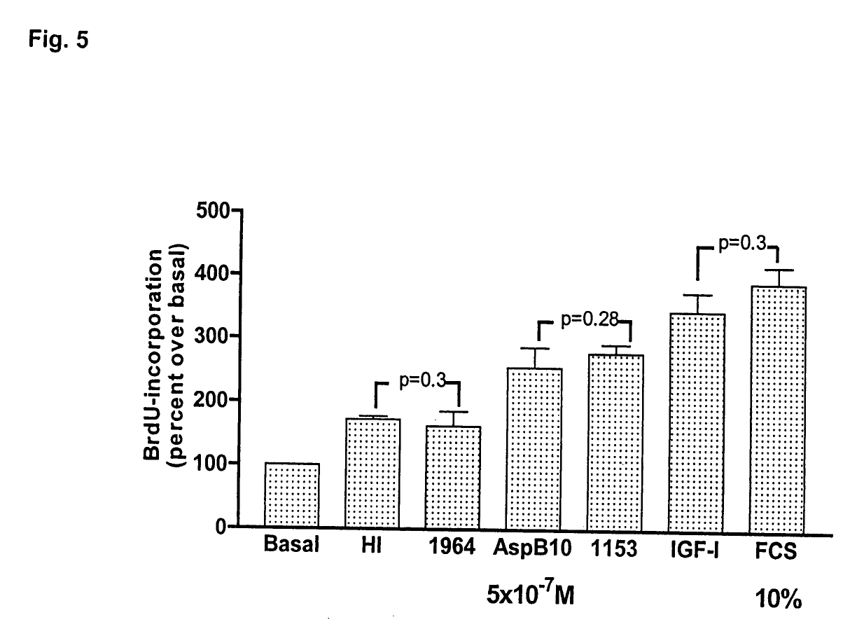

The ERK½ signaling pathway plays a critical role in the regulation of cellular proliferation and differentiation (25). Cellular proliferation of K6 myoblasts was also determined in response to human insulin and the different analogs by monitoring DNA synthesis using the incorporation of bromo-deoxyuridine and a highly sensitive chemiluminescence immunoassay. Serum-starved myoblasts responded with a 4 fold increase in BrdU-incorporation when stimulated with 10% FCS or IGF-I for 16 h (FIG. 5). Both Asp^{B10} insulin and HMR 1153 were equipotent inducing a 2-3 fold increase in DNA synthesis but were significantly less potent that IGF-I. The smallest response (about 50%) was observed for both human insulin and HMR 1964 (FIG. 5). These two molecules were significantly less potent than Asp^{B10} insulin and HMR 1153. Thus, the growth promoting activity of the insulin analog HMR 1964 is identical to that of human insulin and is completely consistent with the insulin-like activation of the Shc/MAP-kinase cascade by this analog.

### Tyrosine Phosphorylation of IRS-½ in Rat and Human Myoblasts

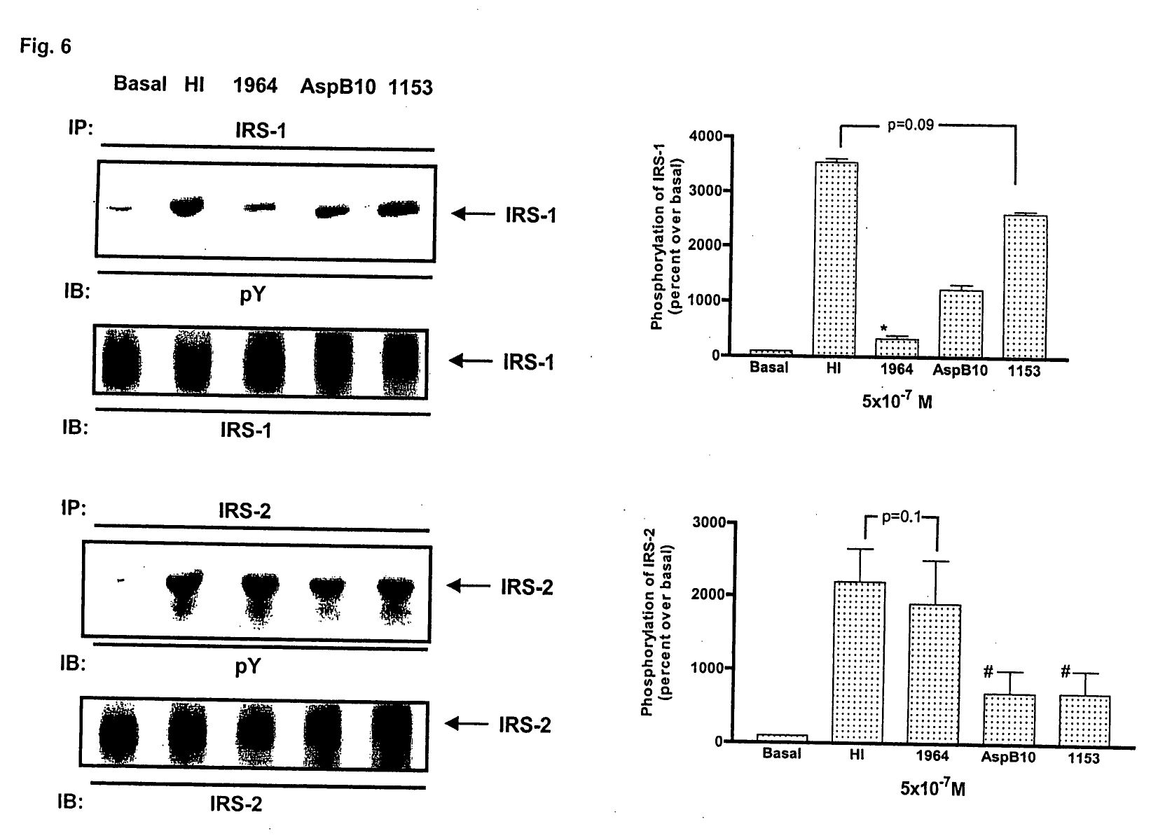

The data obtained demonstrates that the analogs HMR 1153 and Asp^{B10} insulin strongly activate the MAP-kinase pathway via a prominent stimulation of Shc phosphorylation. In contrast, human insulin and the analog HMR 1964 exerted a much weaker effect on this pathway. To further dissect the signaling properties of the different insulin analogs, their effects on the tyrosine phosphorylation of IRS-½ in K6 myoblasts was determined. The cells were stimulated with insulin or insulin analogs for 10 min as described before, and IRS-1 or IRS-2 were immunoprecipitated and immunoblotted with anti-phosphotyrosine antibodies. As shown in FIG. 6, human insulin produced the strongest tyrosine phosphorylation of both IRS-1 and IRS-2, whereas the analogs Asp^{B10} insulin and HMR 1153 induced a less pronounced stimulation of both IRS-proteins. Remarkably, the analog HMR 1964 produced only a marginal phosphorylation of IRS-1, but produced a very strong phosphorylation of IRS-2 that was similar to that seen after stimulation with human insulin (FIG. 6). Quantification of the Western Blots (FIG. 6—right panel) demonstrated an approximately 30 fold and a nearly 20 fold response after treatment with human insulin for IRS-1 and IRS-2, respectively. The analog HMR 1964 exerted a marginal 2 fold effect on the activation of IRS-1, but induced a 20 fold increase of the IRS-2 phosphorylation being as effective as human insulin (FIG. 6).

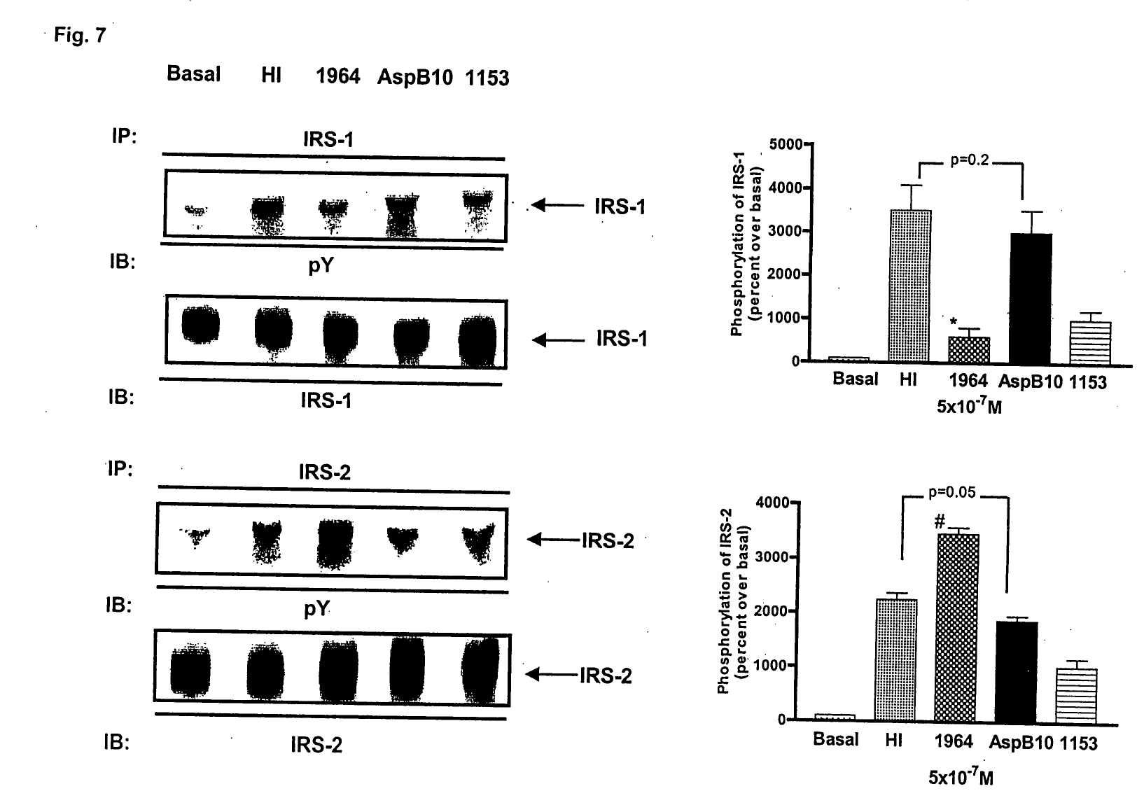

Since the preferred stimulation of IRS-2 by a modified insulin molecule was unexpected, these experiments were repeated in a different cell system. Proliferating primary human skeletal muscle cells were stimulated with human insulin and the different analogs using exactly the same protocol as outlined above for the K6 myoblasts, and the tyrosine phosphorylation of IRS-1 and IRS-2 was analyzed by immunoblotting (FIG. 7). As seen before, human insulin produced a very strong phosphorylation of both IRS-1 and IRS-2 with the analog Asp^{B10} insulin being equipotent to the regular insulin molecule. However, again the insulin analog HMR 1964 produced a marginal phosphorylation of IRS-1 and a very strong tyrosine phosphorylation of IRS-2 that was even significantly higher than that seen after stimulation of cells with human insulin (FIG. 7). Thus, the preferential activation of IRS-2 by the insulin analog HMR 1964 represents a unique property of this molecule that is also effective in human cells.

### Tyrosine Phosphorylation of IRS-½ in Adult Cardiomyocytes

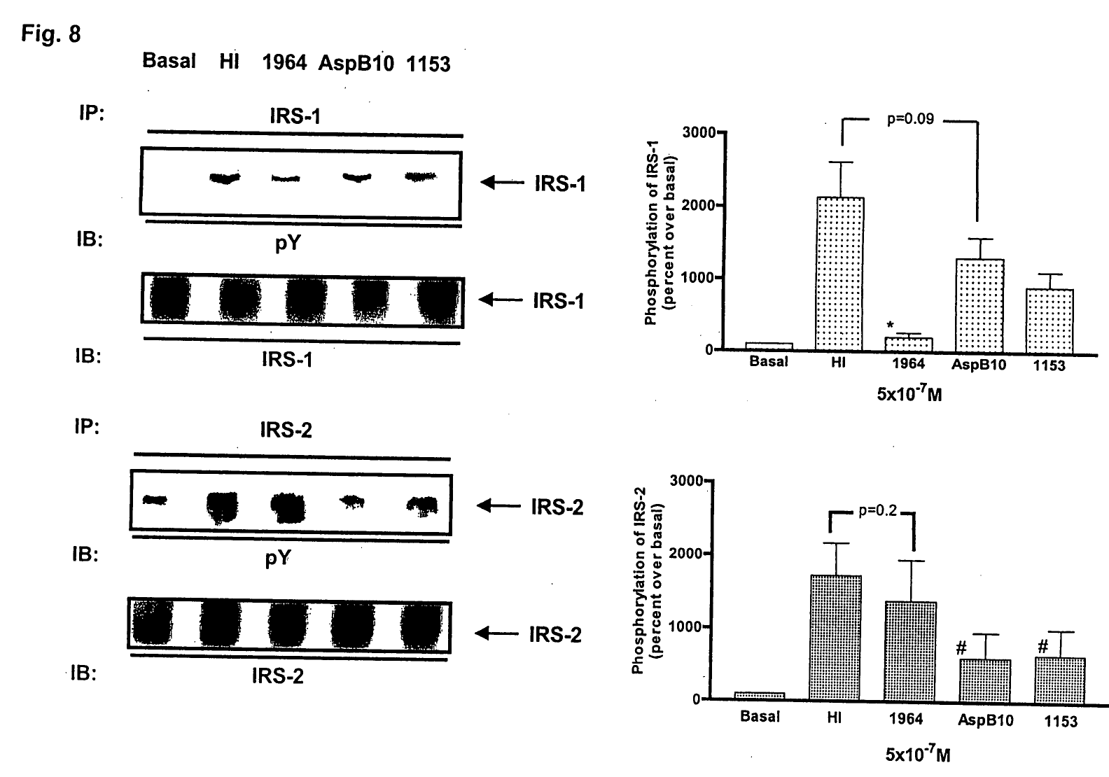

It may be argued that the special effect of HMR 1964 is mediated by the IGF-I receptor and thus could be limited to myoblastic cells. The tyrosine phosphorylation of IRS-1 and IRS-2 in response to the different insulin analogs in primary adult cardiomyocytes was studied. These cells express a high level of insulin receptors but much less IGF-I receptors and have been extensively used for studies on insulin signaling and insulin action (19,26,27). All experiments were conducted under the same conditions as described before for K6 cells and human myoblasts. As shown in FIG. 8, also in this cell system human insulin produced a strong phosphorylation of both IRS-1 and IRS-2, whereas Asp^{B10} insulin and HMR 1153 were less effective. Again, tyrosine phosphorylation of IRS-1 was only marginally activated by HMR 1964 (2 fold). However, this analog produced an 18 fold increase of the tyrosine phosphorylation of IRS-2 reaching the same level as that seen with human insulin (FIG. 8). These data confirm that the novel analog HMR 1964 preferentially signals along the IRS-2 pathway, both in myoblasts and differentiated muscle cells.

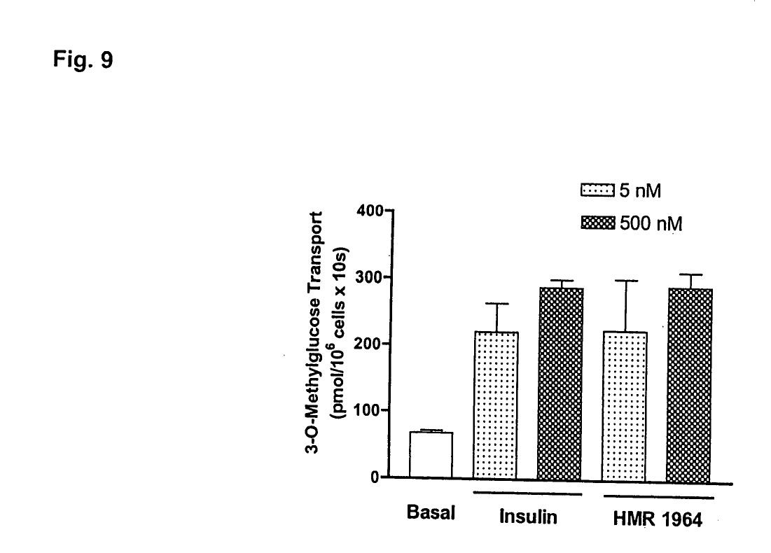

The lack of IRS-1 activation by HMR 1964 may limit the metabolic activity of this analog. To address this issue, the stimulation of 3-O-methylglucose transport by this analog in direct comparison to human insulin using the adult cardiomyocyte system was measured. As presented in FIG. 9, the initial rate of glucose transport was increased 3 and 4.4 fold in response to 5 and 500 nM insulin, respectively. Essentially the same response was observed after treatment of cells with HMR 1964 (FIG. 9). Thus, activation of IRS-2 appears to be sufficient for propagating downstream signaling to glucose transporters in muscle cells.

### Effect of Insulin, Insulin Analogs and IGF-I on Cytokine-Induced Apoptosis in Pancreatic β-Cells

The clonal glucose sensitive rat insulinoma cell line INS-1 is described by M. Asfari et al., Endocrinology 130 (1992) 167-178. Bovine serum albumin (BSA, Fraction V, fatty acid free) was obtained from Boehringer Mannheim (Mannheim, Germany). Fetal calf serum (FCS), Dulbecco's modified Eeagle medium (DMEM), RPMI 1640 medium, non-essential amino acids and penicillin/streptomycin were provided by Gibco (Eggenstein, Germany). The cell death detection ELISA kit was obtained from Roche Diagnostics (Mannheim, Germany). All other chemicals were of the highest grade commercially available.

The rat insulinoma cell line INS-1 was grown in RPMI 1640 medium supplemented with 10% heat-inactivated fetal calf serum (FCS), 500 U/ml penicillin, 50 μg/ml streptomycin, 2 mmol/l glutamine and 50 μmol/l 2-mercaptoethanol and passaged by trypsinization. Functional integrity of the cells was confirmed by measuring glucose-stimulated insulin secretion. INS-1 cells were routinely seeded at 3.5×10⁵ cells/well of a 12-well plate for cell death detection experiments and used on day 4 at 60-70% confluence.

Subconfluent INS-1 cells were incubated for 2 h in RPMI 1640 medium (Eggenstein, Germany) without FCS supplemented with 0.5% BSA. After this serum free period the medium was removed and replaced with fresh RPMI 1640 containing 5% FCS and 0.5% BSA. Cells were then incubated with the cytokine combination interleukin-1β (IL-1β) and interferon-γ (IFN-γ) in the absence or presence of the different insulin peptides (500 nM) or IGF-I (10 nM) for 24 h at 37° C. After removing this medium, the cells were washed twice with ice cold PBS and lysed. The induced apoptosis was measured by the specific determination of mono- and oligonucleosomes in the cytoplasmatic fraction of cell lysates using a cell death detection ELISA kit (Roche). The assay is based on a quantitative sandwich-enzyme-immunoassay-principle using mouse monoclonal anti-histone and anti-DNA peroxidase antibodies. The relative degree of apoptotic cell death was photometrically determined by measuring the peroxidase activity of the immunocomplexes at 405 nm.

In the following table, results are expressed as % inhibition of cytokine-induced apoptosis as measured by nucleosomal release. Data are mean values±SEM of 4-5 separate experiments.

TABLE 1

| | Inhibition of cytokine- |
|---|---|
| Compound | induced apoptosis [%] |
| IGF-1 | 42 ± 2 |
| Human Insulin | 14 ± 3 |
| Asp(B28) Insulin | 13 ± 2 |
| Lys(B28)Pro(B29) Insulin | 26 ± 1 |
| Lys(B3) Glu(B29) Insulin (HMR 1964) | 36 ± 3 |

### DISCUSSION OF RESULTS

It was observed that the novel rapid acting analog Lys^{B3},Glu^{B29} insulin (HMR 1964) was able to generate a preferential activation of the IRS-2 signaling pathway in muscle cells, concomitantly exhibiting the same mitogenic and metabolic properties as regular human insulin. This is the first report on the selective or preferential activation of IRS-2 by an insulin analog. Modification of the B26-B30 region of the insulin molecule has been extensively used to produce insulin analogs with reduced self-association being suitable as rapid acting insulin molecules (3-5). However, modifications within this domain of the insulin molecule are known to increase the affinity of a given analog for the IGF-I receptor, finally leading to an enhanced mitogenic activity and a potential safety risk of these compounds related to long-term use (9,12,15,28). This concept has been reassessed by performing a detailed analysis of the signaling and mitogenic properties of the two analogs LyS^{B3},Glu^{B29} insulin and LyS^{B3},Ile^{B28} insulin (HMR 1153) in rat and human myoblasts expressing a high level of IGF-I receptors. The data suggest that in addition to the binding affinity and the occupancy time at the receptor (14), initial steps of receptor activation and/or processing may also contribute to trigger specific downstream signaling pathways by the insulin molecule.

Both IRS-1 and Shc have been implicated in the activation of the MAP kinase pathway by IGF-I and insulin (29,30), a signalling event with central importance for the control of cellular growth and differentiation by these hormones (31). More recently, a differential interaction of the PTB-domains of Shc and IRS-1 with the IGF-I receptor has been reported (32), and a sustained tyrosine phosphorylation of Shc in response to IGF-I was found to correlate with enhanced MAP kinase activation and growth of human neuroblastoma cells (33), but was unrelated to the tyrosine phosphorylation of IRS-2. As shown by Chow et al. (24), IGF-I receptor internalization is required for signaling via the Shc/MAP kinase pathway, but not the IRS-1 pathway. Consistently, our data show a good correlation between the internalization of the insulin analogs and the activation of the MAP kinase pathway in the K6 myoblasts. Thus, HMR 1153 exhibited a 3-4 fold higher internalization when compared to HMR 1964, and this resulted in a 3-4 fold higher Shc phosphorylation and ERK½ activation in response to HMR 1153. Furthermore, the low rate of internalization of HMR 1964 correlates with a marginal activation of ERK½ by this analog that is even lower than that induced by insulin. It has also been demonstrated that sustained receptor binding decreases endosomal insulin degradation, resulting in enhanced signaling from this intracellular compartment (34). This would explain the strong activation of the MAP kinase by Asp^{B10} insulin, since this analog exhibits a moderate internalization combined with a very low degradation (see Table 2).

Human insulin and HMR 1964 produced a moderate activation of the Shc/MAP kinase pathway and of DNA synthesis in the K6 cells. Also note that under the same conditions insulin induces a prominent activation of both IRS-1 and IRS-2. Thus, the data support the notion that tyrosine phosphorylation of Shc, most likely leading to formation of the Shc-Grb2 complex, represents a key step in IGF-I receptor signaling to the MAP kinase pathway (30,33). It is also evident that the prominent activation of IRS-2 by HMR 1964 does not mediate MAP kinase activation. This is consistent with the view that IRS-2 is of major importance for mediating metabolic events (35-37). Interestingly, the autophosphorylation of the IGF-I receptor in response to HMR 1964 was comparable to that seen after stimulation of cells with Asp^{B10} insulin; however, association of Shc with the IGF-I receptor was much stronger after stimulation with Asp^{B10} insulin. Furthermore, HMR 1153 produced a very strong autophosphorylation of the IGF-I receptor but the same association with Shc when compared to Asp^{B10} insulin. These observations demonstrate that the insulin/receptor interaction may control the specificity of downstream signaling by a combination of several mechanisms. In addition to binding, this may include: i) the internalization and processing of the ligand receptor complex, ii) the half-life of the receptor complex, and iii) a differential phosphorylation pattern of the IGF-I/insulin receptor in response to the different analogs. The latter possibility fits with the observation that Shc and IRS-1 employ functionally distinct mechanisms to recognize tyrosine phosphorylated receptors (32,38).

The mechanism by which ligands interact with their receptors to mediate signaling is still not understood in great detail at the molecular level. The ligand-induced receptor activation has been suggested to involve a conformational switch in the quaternary structure upon ligand binding with movements of the extracellular alpha parts and a congregation of the cytoplasmic tyrosine kinase regions to enable activation (39,40). Thus it may be speculated that differences in the structural changes of the receptor induced by the analogs possibly affect the receptor phosphorylation pattern leading to a differential interaction with downstream substrates and thus resulting in divergent action profiles. Different interactions between the receptor and the analogs could possibly switch the receptor conformation to a state that allows the formation of a stable complex between the receptor and a specific substrate. A hypothetical explanation for the discrepancies in binding potency and receptor phosphorylation obtained by the analogs is that they bind to the receptor in a different manner locking the subunits of the receptors in distinct conformation states and thus affecting its phosphorylation pattern. However, extensive and detailed functional and high resolution structural studies will be necessary to confirm this hypothesis.

Human insulin exerted the most prominent activation of IRS-1 and IRS-2 in both rat and human myoblasts and adult cardiomyocytes. HMR 1153 and Asp^{B10} insulin were much less effective at this level of the insulin signaling cascade despite a 2fold higher proliferative activity of these analogs in the K6 myoblasts. Thus Shc/MAP kinase signaling is the major determinant of the mitogenic activity of insulin analogs. Surprisingly, the analog HMR 1964 produced only a marginal activation of IRS-1 in the three cell systems, but a prominent activation of IRS-2, that was even significantly different from regular insulin in the human myoblasts. This may be a specific property of myoblastic cells expressing a high level of the IGF-I receptor. However, as shown here, HMR 1964 produces an exclusive activation of IRS-2 in adult rat cardiomyocytes, a cell expressing a high level of insulin receptors (26). Furhtermore, the activation of IRS-2 is sufficient to produce a full metabolic response in the adult cardiomyocyte, since HMR 1964 was equipotent to insulin in activating glucose transport in these cells. Consistently, IRS-1 but not IRS-2 has been found to induce the Ras-MAP kinase signaling required for fetal brown adipocyte proliferation (37).

Further, IRS-1 represents the main substrate mediating the mitogenic actions of IGF-I receptors in hepatocytes (36), whereas IRS-2 is a dominant regulator of the metabolic effects of insulin in L6 muscle cells (35). The molecular basis of the preferred activation of IRS-2 by HMR 1964 is presently unknown. An alignment of the predicted amino acid sequence of murine IRS-1 and IRS-2 revealed two conserved regions, the IH1(PH) and the IH2(PTB) domains (41). A third region found in IRS-1, called SAIN domain, is poorly conserved between IRS-1 and IRS-2 (42). Recent studies have identified a novel domain of strong interaction in the central region of IRS-2, localized between amino acids 591 and 786, which is absent in IRS-1. This IRS-2 specific region was found to be independent of the NPX(p)Y-motif. However it requires a functional insulin receptor kinase and the presence of three tyrosine phosphorylation sites in the regulatory loop (Tyr1146, Tyr1150 and Tyr1151). Importantly this novel domain may provide a mechanism by which the stoichiometry of regulatory loop autophosphorylation enhances IRS-2 phosphorylation. These results provide evidence that IRS-2, unlike IRS-1 can interact with tyrosine phosphorylated receptors via multiple independent binding motifs and reveal a novel mechanism regulating the interaction between receptor and IRS-2 that may distinguish the signal of IRS-2 from IRS-1 (43). Further it has previously been shown that Tyr960 is not essential for IRS-2 stimulation, but it is needed for IRS-1 phosphorylation (44). As a result, signaling specificity through the IRS and SHC proteins may be accomplished by distinct phosphorylation patterns during interaction with the activated receptor.

In summary, the mitogenic and metabolic properties of insulin analogs may result from a complex series of initial receptor/ligand interactions involving binding affinity, the timing-dependent specificity, receptor internalization and a specific pattern of receptor phosphorylation. The unique property of HMR 1964 showing a preferred activation of IRS-2 combined with a marginal activation of the MAP kinase mitogenic pathway clearly indicates, that the primary structure of the insulin molecule contains sufficient information to control hormonal action at the downstream level. Selective activation of IRS-2 by an insulin analog may be of central interest for strategies to optimize insulin therapy.

TABLE 2
Binding, internalization and degradation of
insulin and insulin analogs in rat myoblasts

| | | Internalization | |
|---|---|---|---|
| | Binding | (cell-associated | Degradation |
| | (fmol/2 × 10⁵ cells) | cpm) | (%) |
| Human insulin | 2.8 ± 0.06 | 22.0 ± 2 | 6.3 ± 0.5# |
| 1964 | 2.2 ± 0.1 | 16.0 ± 3 | 3.0 ± 0.4 |
| AspB10 | 4.2 ± 0.05* | 34.0 ± 4* | 2.6 ± 0.7 |
| 1153 | 4.4 ± 0.01* | 55.0 ± 11* | 5.0 ± 0.3# |

*significantly different from human insulin and 1964 at p < 0.05;
#significantly different from 1964 and AspB10 at p < 0.05. Data are mean values ± SEM taken from 4-5 separate experiments. Binding, internalization and degradation of insulin and insulin analogs was determined as described in Methods.

### REFERENCES

1. The Diabetes Control and Complications Trial Research Group: The effect of intensive treatment of diabetes on the development and progression of long-term complications in insulin-dependent diabetes mellitus. N Engl J Med 329:977-86, 1993
2. Heinemann L, Starke M, Hohmann A, Berger M: Timing between the subcutaneous administration of insulin and consumption of a carbohydrate rich meal. Horm Metab Res Suppl 26:137-39, 1992
3. Bolli G B, Di Marchi R D, Park G D, Pramming S, Koivisto V A: Insulin analogs and their potential in the management of diabetes mellitus. Diabetologia 42:1151-67, 1999
4. Brange J, Volund A: Insulin analogs with improved pharmacokinetic profiles. Adv Drug Deliv Rev 35:307-35, 1999
5. Vajo Z, Duckworth W C: Genetically engineered insulin analogs: diabetes in the new millenium. Pharmacol Rev 52:1-9, 2000
6. Mosekilde E, Jensen K S, Binder C, Pramming S, Thorsteinsson B: Modeling absorption kinetics of subcutaneous injected soluble insulin. J Pharmacokinet Biopharm 17:67-87, 1989
7. Hildebrandt P, Sejrsen P, Nielsen S L, Birch K, Sestoft L: Diffusion and polymerization determines the insulin absorption from subcutaneous tissue in diabetic patients. Scand J Clin Lab Invest 45:685-90, 1985
8. Brange J, Owens D R, Kang S, Volund A: Monomeric insulins and their experimental and clinical implications. Diabetes Care 13:923-54, 1990
9. Slieker L J, Brooke G S, DiMarchi R D, Flora D B, Green L K, Hoffmann J A, Long H B, Fan L, Shields J E, Sundell K L, Surface P L, Chance R E: Modifications in the B10 and B26-30 regions of the B chain of human insulin alter affinity for the human IGF-I receptor more than for the insulin receptor. Diabetologia 40:S54-61, 1997
10. Drejer K: The bioactivity of insulin analogs from in vitro receptor binding to in vivo glucose uptake. Diabetes Metab Rev 8: 259-85, 1992
11. Hansen B F, Danielsen G M, Drejer K, Sorensen A R, Wiberg F C, Klein H H, Lundemose A G: Sustained signalling from the insulin receptor after stimulation with insulin analogs exhibiting increased mitogenic potency. Biochem J 315:271-79, 1996
12. Kurtzhals P, Schaffer L, Sorensen A, Kristensen C, Jonassen I, Schmid C, Trub T: Correlations of receptor binding and metabolic and mitogenic potencies of insulin analogs designed for clinical use. Diabetes 49:999-1005, 2000
13. Bahr M, Kolter T, Seipke G, Eckel J: Growth promoting and metabolic activity of the human insulin analog [GlyA21,ArgB31,ArgB32]insulin (HOE 901) in muscle cells. Eur J Pharmacol 320:259-65, 1997
14. Shymko R M, Dumont E, De Meyts P, Dumont J E: Timing-dependence of insulin-receptor mitogenic versus metabolic signalling: a plausible model based on coincidence of hormone and effector binding. Biochem J 339:675-83, 1999
15. Milazzo G, Sciacca L, Papa V, Goldfine I D, Vigneri R: ASPB10 insulin induction of increased mitogenic responses and phenotypic changes in human breast epithelial cells: evidence for enhanced interactions with the insulin-like growth factor-I receptor. Mol Carcinog 18:19-25, 1997
16. Dransfeld O, Uphues I, Sasson S, Schurmann A, Joost H G, Eckel J: Regulation of subcellular distribution of GLUT4 in cardiomyocytes: Rab4A reduces basal glucose transport and augments insulin responsiveness. Exp Clin Endocrinol Diabetes 108:26-36, 2000
17. Eckel J, Pandalis G, Reinauer H: Insulin action on the glucose transport system in isolated cardiocytes from adult rat. Biochem J 212:385-92, 1983
18. Eckel J, Reinauer H: Involvement of hormone processing in insulin-activated glucose transport by isolated cardiac myocytes. Biochem J 249:111-16, 1988
19. Till M, Ouwens D M, Kessler A, Eckel J: Molecular mechanisms of contraction-regulated cardiac glucose transport. Biochem J 346:841-47, 2000
20. Kolter T, Uphues I, Eckel J: Molecular analysis of insulin resistance in isolated ventricular cardiomyocytes of obese Zucker rats. Am J Physiol 273:E59-67, 1997
21. Slieker L J, Brooke G S, DiMarchi R D, Flora D B, Green L K, Hoffmann J A, Long H B, Fan L, Shields J E, Sundell K L, Surface P L, Chance R E: Modifications in the B10 and B26-30 regions of the B chain of human insulin alter affinity for the human IGF-I receptor more than for the insulin receptor. Diabetologia 40:S54-61, 1997
22. Drejer K, Kruse V, Larsen U D, Hougaard P, Bjorn S, Gammeltoft S: Receptor binding and tyrosine kinase activation by insulin analogs with extreme affinities studied in human hepatoma HepG2 cells. Diabetes 40:1488-95, 1991
23. Di Guglielmo G M, Drake P G, Baass P C, Authier F, Posner B I, Bergeron J J: Insulin receptor internalization and signalling. Mol Cell Biochem 182:59-63, 1998
24. Chow J C, Condorelli G, Smith R J: Insulin-like growth factor-I receptor internalization regulates signaling via the Shc/mitogen-activated protein kinase pathway, but not the insulin receptor substrate-1 pathway. J Biol Chem 273:4672-80, 1998
25. Alessi D R, Saito Y, Campbell D G, Cohen P, Sithanandam G, Rapp U, Ashworth A, Marshall C J, Cowley S: Identification of the sites in MAP kinase kinase-1 phosphorylated by p74raf-1. EMBO J 13:1610-19, 1994
26. Eckel J, Reinauer H: The insulin receptor of adult heart muscle cells. Basic Res Cardiol 80: 61-64.1985
27. Kessler A, Uphues I, Ouwens D M, Till M, Eckel J: Diversification of cardiac insulin signaling involves the p85 alpha/beta subunits of phosphatidylinositol 3-kinase. Am J Physiol Endocrinol Metab 280:E65-74, 2001
28. Bornfeldt K E, Gidlof R A, Wasteson A, Lake M, Skottner A, Arnqvist H J: Binding and biological effects of insulin, insulin analogs and insulin-like growth factors in rat aortic smooth muscle cells. Comparison of maximal growth promoting activities. Diabetologia 34:307-13, 1991
29. Baltensperger K, Kozma L M, Cherniack A D, Klarlund J K, Chawla A, Banerjee U, Czech M P: Binding of the Ras activator son of sevenless to insulin receptor substrate-1 signaling complexes. Science 260:1950-52, 1993
30. Ouwens D M, van der Zon G C, Pronk G J, Bos J L, Moller W, Cheatham B, Kahn C R, Maassen J A: A mutant insulin receptor induces formation of a Shc-growth factor receptor bound protein 2 (Grb2) complex and p21 ras-GTP without detectable interaction of insulin receptor substrate 1 (IRS1) with Grb2. Evidence for IRS1-independent p21 ras-GTP formation. J Biol Chem 269:33116-22, 1994
31. Pelech S L, Sanghera J S: Mitogen-activated protein kinases: versatile transducers for cell signaling. Trends Biochem Sci 17:233-38, 1992
32. Tartare-Deckert S, Sawka-Verhelle D, Murdaca J, Van Obberghen E: Evidence for a differential interaction of SHC and the insulin receptor substrate-1 (IRS-1) with the insulin-like growth factor-I (IGF-I) receptor in the yeast two-hybrid system. J Biol Chem 270:23456-60, 1995
33. Kim B, Cheng H L, Margolis B, Feldman E L: Insulin receptor substrate 2 and Shc play different roles in insulin-like growth factor I signaling. J Biol Chem 273:34543-50, 1998
34. Bevan A P, Seabright P J, Tikerpae J, Posner B I, Smith G D, Siddle K: The role of insulin dissociation from its endosomal receptor in insulin degradation. Mol Cell Endocrinol 164:145-57, 2000
35. Miele C, Caruso M, Calleja V, Auricchio R, Oriente F, Formisano P, Condorelli G, Cafieri A, Sawka-Verhelle D, Van Obberghen E, Beguinot F: Differential role of insulin receptor substrate (IRS)-1 and IRS-2 in L6 skeletal muscle cells expressing the Arg1152-->Gln insulin receptor. J Biol Chem 274:3094-102, 1999
36. Rother K I, Imai Y, Caruso M, Beguinot F, Formisano P, Accili D: Evidence that IRS-2 phosphorylation is required for insulin action in hepatocytes. J Biol Chem 273:17491-97, 1998
37. Valverde A M, Lorenzo M, Pons S, White M F, Benito M: Insulin receptor substrate (IRS) proteins IRS-1 and IRS-2 differential signaling in the insulin/insulin-like growth factor-I pathways in fetal brown adipocytes. Mol Endocrinol 12:688-97, 1998
38. Farooq A, Plotnikova O, Zeng L, Zhou M M: Phosphotyrosine binding domains of Shc and insulin receptor substrate 1 recognize the NPXpY motif in a thermodynamically distinct manner. J Biol Chem 274:6114-21, 1999
39. McDonald N Q, Murray-Rust J, Blundell T L: The first structure of a receptor tyrosine kinase domain: a further step in understanding the molecular basis of insulin action. Structure 3:1-6, 1995
40. Hubbard S R, Wei L, Ellis L, Hendrickson W A: Crystal structure of the tyrosine kinase domain of the human insulin receptor. Nature 372:746-54, 1994
41. Sun X J, Wang L M, Zhang Y, Yenush L, Myers M G Jr, Glasheen E, Lane W S, Pierce J H, White M F: Role of IRS-2 in insulin and cytokine signalling. Nature 377:173-77, 1995
42. O'Neill T J, Craparo A, Gustafson T A: Characterization of an interaction between insulin receptor substrate 1 and the insulin receptor by using the two-hybrid system. Mol Cell Biol 14:6433-42, 1994
43. Sawka-Verhelle D, Baron V, Mothe I, Filloux C, White M F, Van Obberghen E: Tyr624 and Tyr628 in insulin receptor substrate-2 mediate its association with the insulin receptor. J Biol Chem 272:16414-20, 1997
44. Chaika O V, Chaika N, Volle D J, Hayashi H, Ebina Y, Wang L M, Pierce J H, Lewis R E: Mutation of tyrosine 960 within the insulin receptor juxtamembrane domain impairs glucose transport but does not inhibit ligand-mediated phosphorylation of insulin receptor substrate-2 in 3T3-L1 adipocytes. J Biol Chem 274:12075-80, 1999

What is claimed is:

1. A method of protecting beta cell mass of a patient, which comprises administering to the patient an effective amount of LYS^{B3}, Glu^{B29} insulin.

2. The method of claim 1, wherein said patient is Type II diabetic.

3. The method of claim 2, wherein said Type II diabetic patient is impaired glucose tolerant and/or has impaired fasting glucose.

4. The method of claim 1, wherein the ability of the beta cells of said patient to produce insulin has been impaired.

5. The method of claim 1, wherein said patient has autoimmune deficiencies.

6. The method of claim 1, wherein said patient is obese, insulin resistant and/or hyperinsulinemic.

7. A method of protecting beta cell function of a patient, which comprises administering to the patient an effective amount of LyS^{B3}, Glu^{B29} insulin.

8. A method of rejuvinating beta cell mass of a patient, which comprises administering to the patient an effective amount of LyS^{B3}, Glu^{B29} insulin.

9. A method of rejuvinating beta cell function of a patient, which comprises administering to the patient an effective amount of LyS^{B3}, Glu^{B29} insulin.

10. A method of inhibiting apoptosis in pancreatic beta-cells of a patient, which comprises administering to the patient an effective amount of LyS^{B3}, Glu^{B29} insulin.

11. A method of activating insulin receptor substrate-2 to protect at least one property chosen from beta cell mass and beta cell function, which comprises administering to the patient an effective amount of LyS^{B3}, Glu^{B29} insulin.

12. A pharmaceutical composition comprising Lys^{B3}, Glu^{B29} insulin in an amount effective to protect against loss of beta cell mass, protect against loss of beta cell function, rejuvenate beta cells mass or rejuvenate beta cell function or any combination thereof without corresponding significant reduction in blood glucose levels.

13. A pharmaceutical composition consisting essentially of LyS^{B3}, Glu^{B29} insulin, wherein said LyS^{B3}, Glu^{B29} insulin is present in an amount ranging from about 0.01 IU/kg to about 0.1 IU/kg of the body weight of a patient.

14. A pharmaceutical composition comprising LyS^{B3}, Glu^{B29} insulin with the proviso that said pharmaceutical composition does not contain human insulin, wherein said LyS^{B3}, Glu^{B29} insulin is present in an amount ranging from about 0.01 IU/kg to about 0.1 IU/kg of the body weight of a patient.
